(19) United States
(12) Patent Application Publication
(10) Pub. No.: US 2003/0176803 A1
(43) Pub. Date: Sep. 18, 2003

(54) Personal breath tester

(76) Inventor: Edward Gollar, Cincinnati, OH (US)

Correspondence Address: DINSMORE & SHOHL, LLP, 1900 CHEMED CENTER, 255 EAST FIFTH STREET, CINCINNATI, OH, 45202, US

(21) Appl. No.: 10/097,460

(22) Filed: Mar. 14, 2002

(51) Int. Cl. A61B 5/08

(52) U.S. Cl. 600/532

(57) ABSTRACT

Apparatus for detecting blood alcohol level in breath. Apparatus has a breath channel; and electrochemical fuel cell in communication with the breath channel; a temperature sensor in communication with the breath channel; a processor in communication with the temperature sensor and electrochemical fuel cell; and a computer readable storage medium containing executable instructions for the processor.

### TECHNICAL FIELD

The present invention relates to the field of gaseous breath detection systems, and methods for using the same, and more particularly, to the field of portable personal gaseous breath detection apparatus and methods for using same.

### BACKGROUND OF THE INVENTION

There are several methods for determining the alcohol content (or level) of a person's breath. A common method is to use a tin-oxide semiconductor alcohol sensor. It has the advantage of low cost at the expense of accuracy, alcohol specificity, and electrical power consumption. Another method is to employ the use of an electrochemical fuel cell alcohol sensor. While this type of sensor tends to be more accurate, more alcohol specific, and utilizes less electrical power, the sensor itself is significantly more expensive and has traditionally required the use of an active sampling mechanism such as a pump. The pump adds cost and size to the device, and utilizes electrical power. Both methods also typically require the use of a pressure sensor to determine when the user is blowing into the device.

Accordingly, it is desirable to have a breath detection apparatus that utilizes an electrochemical fuel cell alcohol sensor for accuracy, alcohol specificity, and low power consumption, and eliminates the need for a sampling mechanism, saving more in cost, power consumption, and size. Furthermore, it is desirable to have such breath detection apparatus with the traditional pressure sensor eliminated in favor of a configuration that utilizes a temperature sensor as a flow sensor, thus saving in the size and cost of the device.

### SUMMARY OF THE INVENTION

Accordingly, it is an object of the present invention to provide an improved breath alcohol tester. In particular, it is a benefit of the present invention to provide a breath alcohol tester that combines low cost, small size, low power consumption, and alcohol specificity.

One embodiment of the present invention comprises an apparatus for detecting gaseous component levels in breath. The apparatus comprises: a breath channel; an electrochemical sensor in fluid communication with the breath channel; a processor in electrical communication with the electrochemical sensor; and computer readable storage medium in electrical communication with the processor, wherein the computer readable storage medium contains executable instructions for the processor; wherein the apparatus is configured to calculate approximate gaseous components levels in a breath without utilizing a sampling pump.

Another embodiment of the present invention comprises a breath detection apparatus for detecting gaseous component levels in breath. The apparatus comprises: a gas sensor; a processor in electrical communication with the gas sensor; and a computer readable storage medium in electrical communication with the processor, wherein the computer readable storage medium contains executable instructions for the processor; and a wireless transmitter; wherein the wireless transmitter transmits a signal to an external receiver.

Yet another embodiment of the present invention comprises an apparatus for detecting gaseous component levels in breath. The apparatus comprises: a breath channel; a gas sensor in fluid communication with the breath channel; a processor in electrical communication with the gas sensor; computer readable storage medium in electrical communication with the processor, wherein the computer readable storage medium contains executable instructions for the processor; and temperature sensor in fluid communication with the breath channel; wherein the temperature sensor is utilized to determine breath flow rate.

Still another embodiment of the present invention comprises a method for detecting gaseous component levels in breath, The method comprises: obtaining an initial signal from a temperature sensor; monitoring the temperature sensor for a temperature change; calculating airflow rate utilizing the temperature sensor signal; and calculating gaseous component levels in breath utilizing airflow rate.

Yet another embodiment of the present invention comprises a method for detecting gaseous component levels in breath. The method comprises: receiving a breath stream in a breath channel; obtaining a signal from an electrochemical sensor; and calculating a gaseous component level in breath utilizing the electrochemical sensor.

One embodiment of the present invention comprises an apparatus for detecting gaseous component levels in breath. The apparatus comprises: a breath passage having a flowpath, a proximal end and a distal end, wherein the proximal end comprises an inlet for accepting a person's breath and the distal end comprises an outlet for venting the breath; a temperature sensor in fluid communication with the flowpath; an electrochemical sensor in fluid communication with the flowpath; a processor in electrical communication with the temperature sensor and the electrochemical sensor; and a computer readable storage medium in electrical communication with the processor, wherein the computer readable medium contains executable instructions for the processor; wherein the apparatus is configured to approximate gaseous component level in the breath without utilizing a sampling pump.

Another embodiment of the present invention comprises an apparatus for detecting gaseous component level in breath. The apparatus comprises: a breath channel; a gas sensor in fluid communication with the breath channel; a processor in electrical communication with the gas sensor; a computer readable storage medium in electrical communication with the processor, wherein the computer readable storage medium contains executable instructions for the processor; and wherein the apparatus is configured to approximate gaseous component level in a breath without utilizing a sampling pump; and further wherein the apparatus is configured to cease functioning at a pre-determined time.

Yet another embodiment of the present invention comprises an apparatus for detecting gaseous component level in breath. The apparatus comprises: a breath channel; a gas sensor in fluid communication with the breath channel; a processor in electrical communication with the gas sensor; a computer readable storage medium in electrical communication with the processor, wherein the computer readable storage medium contains executable instructions for the processor; and wherein the apparatus is configured to approximate gaseous component level in a breath without utilizing a sampling pump; and further wherein the apparatus is configured to cease functioning outside a pre-determined temperature range.

One embodiment of the present invention comprises an ignition interlock system. The system comprises: the breath detection apparatus and a wireless receiver; a computing device in electrical communication with the wireless receiver; a computer readable storage medium in electrical communication with the computing device, wherein the computer readable medium contains executable instructions for the computing device; a switch in electrical communication with the computing device and ignition control line of a vehicle; wherein the wireless receiver is configured to receive signals from the breath detection apparatus.

Yet another embodiment of the present invention comprises an identification system for a breath detection interlock system. The system comprises: a wireless transmitter and receiver; a processor in electrical communication with the transmitter and receiver; a computer readable medium in electrical communication with the processor, wherein the computer readable storage medium contains executable instructions for the processor; and wherein the executable instruction comprise instructions to maintain a continuous signal between the transmitter and receiver.

Still another embodiment of the present invention comprises an identification method for a breath detection system. The method comprises: confirming a user's identity; maintaining a continuous signal between the transmitter and receiver after the identity has been confirmed; and if the signal between the transmitter and receiver is not continuous, aborting the breath detection system and restart the system.

Another embodiment of the present invention comprises an identification system for a breath detection interlock system. The system comprises: a passive infrared detector; a processor in electrical communication with the detector; a computer readable medium in electrical communication with the processor, wherein the computer readable storage medium contains executable instructions for the processor; and wherein the executable instruction comprise instructions to monitor infrared signals utilizing the passive infrared detector.

Yet another embodiment of the present invention is an identification method for a breath detection system. The method comprises: confirming a user's identity; maintaining a continuous signal between the user and the passive infrared detector; and if the signal received by the passive infrared detector is not continuous, aborting the breath detection system and restart the system.

Another embodiment of the present invention is an identification system for a breath detection interlock system. The system comprises: a motion sensor; a processor in electrical communication with the motion sensor; a computer readable medium in electrical communication with the processor, wherein the computer readable storage medium contains executable instructions for the processor; and wherein the executable instruction comprise instructions to monitor the movement of the motion sensor.

Still yet another embodiment of the present invention is an identification method for a breath detection system. The method comprises: confirming a user's identity; monitoring a motion sensor output; and if the signal received by the motion sensor exceeds a pre-determined threshold, abort the breath detection system and restart the system.

Another embodiment of the present invention is an identification method for a breath detection system. The method comprises: confirming a user's identity; and initiating a countdown timer executable instructions, wherein if a breath test has not been initiated by the lapse of the count down timer, abort the breath detection system and restart the system.

In one aspect of the present invention, when the user blows into the device, a temperature sensor which is connected to a controller and is situated in the breath channel portion of the device detects that the user is blowing and at what flow rate. The breath channel is also directly connected to a electrochemical fuel cell ethanol sensor that gives an electrical output when it is exposed to ethanol in the breath. The positioning of the ethanol sensor directly in the breath channel eliminates the need for a mechanical sampling system. The ambient temperature of the device is determined by the controller from the breath temperature sensor before the user starts blowing. After the user has stopped blowing, an algorithm contained within the controller can calculate the user's breath alcohol content by taking into account the flow rate, the length of time the user was blowing, and the temperature of the ethanol sensor.

In another aspect of the present invention, one or more safety mechanisms, prevent the device from giving an erroneous reading. The controller can shut down the device to prevent the user from taking a test if the ambient temperature is outside of a range within which the ethanol sensor can give an accurate reading. The controller can also shut down the device if the length of time that has expired since the device was constructed and calibrated is such that the output of the ethanol sensor has drifted and will no longer give an accurate reading.

In another aspect of the present invention, the system further includes a breath alcohol ignition interlock device. In this embodiment, a wireless transmitter is incorporated into the controller circuit. A physically separate controller which contains a wireless receiver is installed in the vehicle and attached to the vehicle's ignition circuit. After the user takes a breath test, the transmitter sends a signal to the receiver in the controller in the vehicle, which allows the vehicle to start if the breath alcohol level is below a predetermined level, and not allowing the vehicle to start otherwise.

The present invention also provides a method for using the breath tester as an ignition interlock for the consumer market. A separate wireless transmitter is used that allows the supervising agent (whether it be the parent, spouse, etc.) to enable the vehicle's ignition without having to take a breath test. This transmitter also allows the supervising agent to program the device with various options.

Another aspect of the present invention provides an ignition interlock for the court-mandated market. A voice recognition circuit is employed in the breath tester that requires the user to speak one or more words into the device before taking the breath test. If the controller in the device matches the spoken words to those that were previously stored in the device when it was trained by the intended user during installation, then a subsequent breath test will be allowed. If the words are not matched, then a breath test is not allowed. To insure that the device cannot be passed to another individual after the words are spoken by the intended user, several methods may be employed: allowing a short interval of time between the spoken words and the breath test; using a transmitter and receiver combination that bounces energy off the user's face and detects when the transmitted beam is interrupted; using a motion sensor that detects if the device is moved quickly; using an infrared heat sensor that detects a change in sensed body heat.

Still other objects and advantages of the present invention will become apparent to those skilled in this art from the following description wherein there is shown and described exemplary embodiments of this invention, including a best mode currently contemplated for the invention, simply for purposes of illustration. As will be realized, the invention is capable of other different aspects and embodiments without departing from the scope of the invention.

### BRIEF DESCRIPTION OF THE DRAWINGS

While the specification concludes with claims particularly pointing out and distinctly claiming the present invention, it is believed the same will be better understood from the following description taken in conjunction with the accompanying drawings in which:

FIG. 1 is an operational block diagram of a breath alcohol tester apparatus in accordance with the present invention;

FIG. 2 is an operational block diagram of an interlock ignition system in accordance with the present invention;

FIG. 3 is an operational block diagram of a wireless master transmitter in accordance with the present invention;

FIG. 4 is a flowchart depicting an exemplary embodiment of the method of detecting breath alcohol levels;

FIG. 5 is a flowchart depicting and exemplary embodiment of voice verification method in accordance with the present invention; and

FIG. 6 is a flowchart depicting an exemplary embodiment of another method in accordance with the present invention.

### DETAILED DESCRIPTION OF THE INVENTION

Reference will now be made in detail to the present exemplary embodiments of the invention, examples of which are illustrated in the accompanying drawings, wherein like numerals indicate the same elements throughout the views.

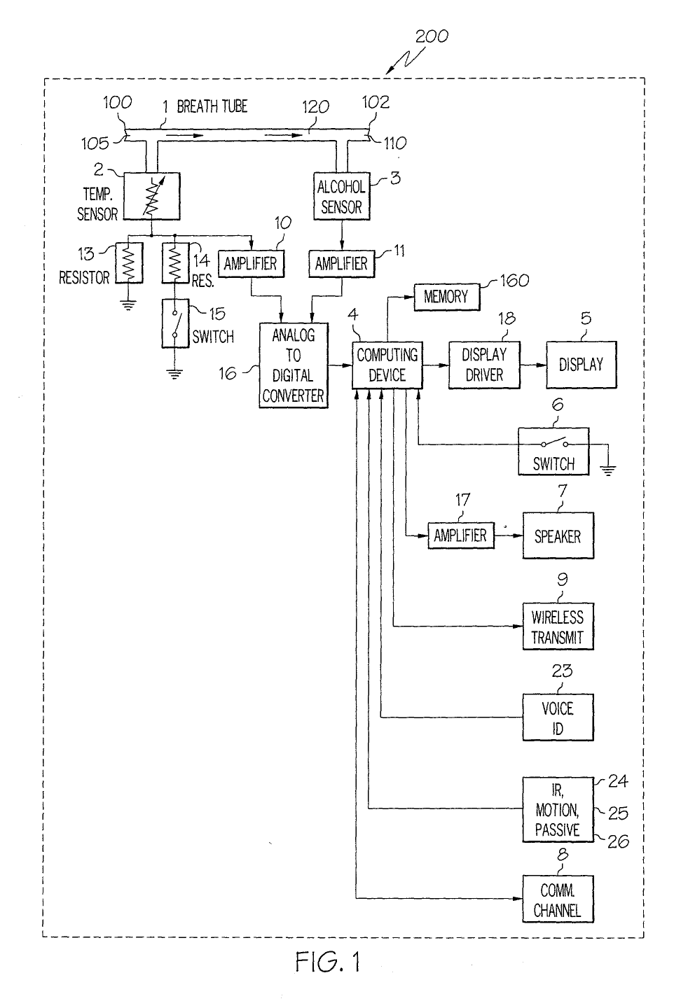

Referring to FIG. 1, the personal breath tester 200 comprises a breath passage 1 having a flowpath 120, a proximal end 100 and a distal end 102, wherein the proximal end 100 comprises an inlet 105 for accepting a person's breath and the distal end 102 comprises an outlet 110 for venting the breath. A temperature sensor 2 is in fluid communication with the flowpath 120 of the breath passage 1. In addition, an alcohol sensor 3 is in fluid communication with the flowpath 120 of the breath passage 1. In an exemplary embodiment, the temperature sensor 2 and/or alcohol sensor 3 are physically contained within the flowpath 120 of the breath passage 1. Since the alcohol sensor 3 is in fluid communication with the flowpath 120, the need for a mechanical pump or sampling system is eliminated.

In one exemplary embodiment, the temperature sensor 2 comprises a thermistor sensor and the alcohol sensor 3 comprises an electrochemical fuel cell with an ethanol sensor. The temperature sensor 2 is in electrical communication with two resistors 13 and 14. The resistor 14 is in electrical communication with an electrical switch 15, which in turn is in electrical communication with a computing device 4. The temperature sensor 2 is also in electrical communication to an amplifier 10 for generating a signal representative of flow rate. The output signal of the flow amplifier 10 is in electrical communication with the analog-to-digital converter 16, which converts the output signal into a digital number that can be interpreted by the computing device 4, such as a microprocessor.

The alcohol sensor 3 is in electrical communication with an amplifier 11. The output signal of the amplifier is in electrical communication with the analog-to-digital converter 16, which converts the output signal into a digital number. The output signal of the analog-to-digital converter is connected to the computing device 4.

A display 5, which in one exemplary embodiment comprises an alphanumeric display, is driven by a display driver circuit 18. The display driver circuit 18 is in electrical communication and is controlled by the computing device 4. In another exemplary embodiment, the present invention further comprises a speaker 7, which is controlled by an amplifier 17, wherein the amplifier is controlled by the computing device 4. A momentary switch 6 and a communication channel 8 are in electrical communication with the computing device 4.

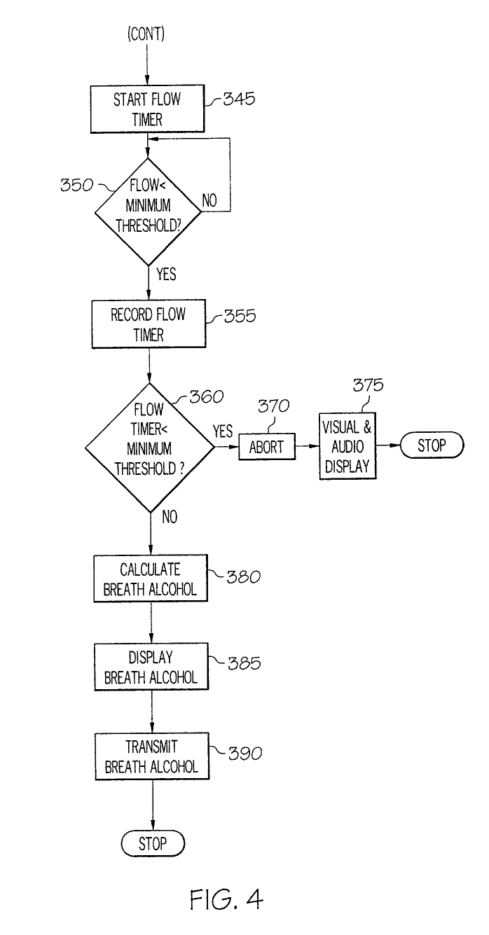

In one exemplary embodiment of the present invention depicted by FIG. 4, a breath test is initiated when a person depresses the switch 6 (step 305) of the personal breath tester 200. When the computing device 4 determines that the switch 6 has been depressed, the computing device 4 obtains the initial temperature of the temperature sensor 2 by opening the switch 15, converting the temperature sensor 2 output signal into a digital number with the analog-to-digital converter 16, and recording that number as the starting value of the temperature sensor 2 (step 310). If the recorded starting value of the temperature sensor 2 is less than 32° C. or greater than 36° C., the switch 15 is left open and the personal breath tester 200 is ready to begin testing breath samples. If the recorded starting value is equal to or more than 32° C. and less than or equal to 36° C. (step 315), then switch 15 is turned on (closes circuit) by the computing device 4 (step 320) to increase the temperature level to that greater than expected human breath (i.e. 34° C.).

When switch 15 is turned on, the resistor 14 is placed in electrical communication with the temperature sensor 2, causing a significant increase in current to flow through the temperature sensor 2. After a short amount of time, this causes heating of the temperature sensor 2, and the internal temperature will rise significantly above 34° C.

Once a suitable initial temperature has been obtained (i.e. less than 32° C. or greater than 36° C.), whether switch 15 is on or off, a person blows into the breath passage 1 of the personal breath detector 200. The temperature of the person's breath is typically 34° C. The stream of air blown into the breath passage will cause the temperature of the temperature sensor 2 to change.

If the initial temperature of the temperature sensor 2 immediately before blowing is below 32° C., then the temperature will rise with blowing. Similarly, if the initial temperature of the temperature sensor 2 is above 36° C., then the temperature will fall with blowing.

This change in temperature is amplified by the flow amplifier 10, converted into a digital signal by the analog-to-digital converter 16, and then sent to the computing device 4. The change in temperature is an indication that the user is blowing, and the rate at which this temperature change occurs is an indication of the flow rate (step 325). A quick change in temperature indicates a higher flow rate than a slow change in temperature. Once the computing device 4 detects that the user is blowing, it converts the alcohol sensor amplifier 11 output into a digital number by way of the analog-to-digital converter 16, and records that number as the baseline value of the alcohol sensor 3 (step 328). In an exemplary embodiment, the baseline value is stored in a computer readable memory unit 160.

The computing device 4 calculates the flow rate (step 330) and compares it to a minimum flow threshold value, which is stored in the computing device or computer readable memory unit 160. If the flow rate is higher than the minimum (step 335), then the computing device 4 starts an internal flow timer (step 345). Once the person stops blowing air into the breath passage and/or the air flow rate drops below the minimum threshold value (step 350), then the computing device 4 records the flow timer value as an indication of how long the person was blowing air into the breath passage at an acceptable rate (i.e. above minimum threshold value) (step 355). If the recorded flow timer value is less than a minimum timer threshold value (step 360), stored in the computing device, then the computing device 4 aborts the breath test (step 370), and sends a visual abort indication to the user. In one exemplary embodiment, the abort indication is a visual indication on the personal breath tester (i.e., such as a display 5). In another exemplary embodiment, the abort indicator is an audible signal through a speaker 7 (step 375). If the recorded flow timer value is less than the minimum timer threshold another breath test must be initiated by the person. The minimum flow rate and flow timer threshold values exist to insure that the person taking the test is providing a minimum volume of deep-lung (alveolar) air into the device.

As long as the minimum flow rate and flow timer threshold values are exceeded, the computing device 4 calculates the blood alcohol level (step 380). In one exemplary embodiment, the fuel cell alcohol sensor sends a signal to the amplifier 11. The amplifier 11 sends an amplified signal to the analog/digital converter 16. The analog/digital converter 16 sends the digital signal to the computing device 4. The computing device 4 retrieves from a computer readable memory storage unit 160, the previously recorded baseline value for the alcohol sensor. The computing device 4 then calculates an equivalent breath alcohol level using an algorithm incorporating the baseline value, the flow rate, the length of time blowing, the temperature of temperature sensor 2 and a calibration factor accounting for variations in output from sensor to sensor. The breath alcohol level is then indicated on the display 5 as a digital number (step 385), along with an audible indication on speaker 7 that the test is completed.

If the embodiment includes an ignition interlock device, the computing devices would then transmit the level and/or a signal to the ignition interlock system (step 390).

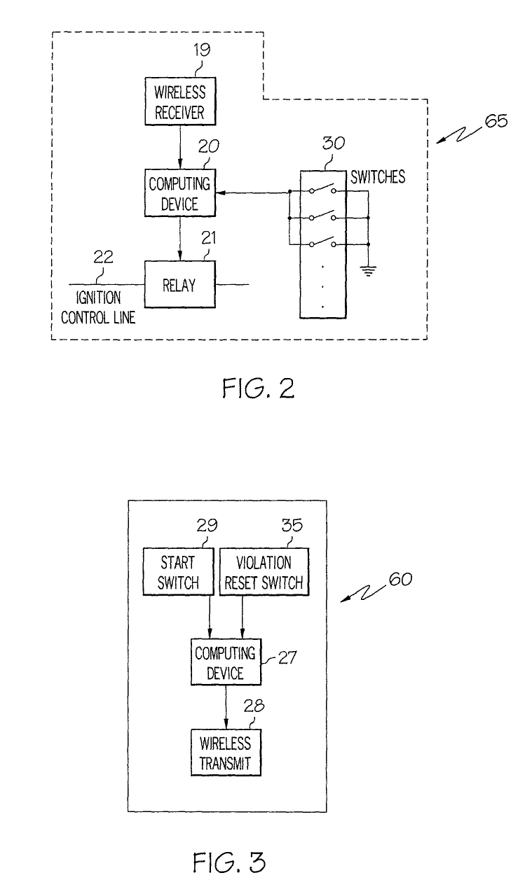

FIG. 2 depicts an exemplary ignition interlock system. The ignition interlock system 65 is located in the vehicle, and contains a computing device 20, a wireless receiver 19, and a relay 21 that controls the vehicle's ignition circuit 22. When the receiver 19 receives the breath alcohol level from the personal breath tester 200, the computing device 20 compares the breath alcohol level to a stored predetermined level. If the received level is below the predetermined level, the relay 21 is engaged by the computing device 20, allowing the ignition control line 22 to enable starting of the vehicle. If the received level is at or above the predetermined level, then the relay is not engaged and the vehicle will not start.

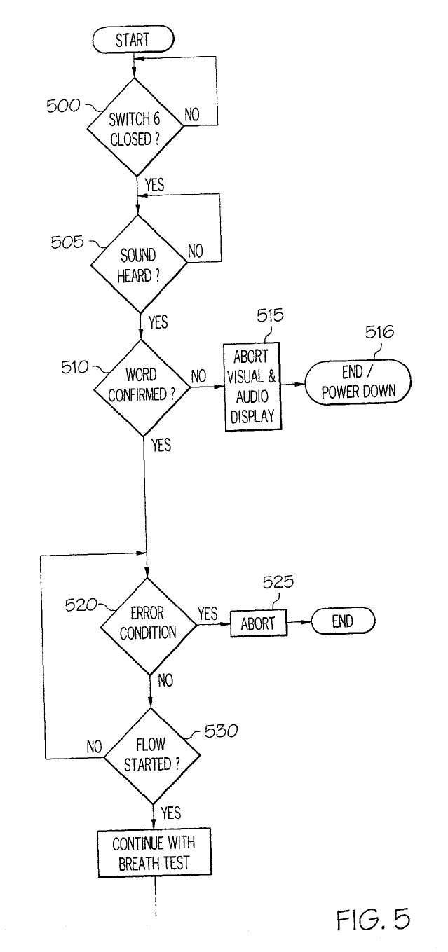

In another exemplary embodiment depicted in FIG. 5, the personal breath tester is used as an ignition interlock device for a court-mandated market. When the user depresses switch 6 (step 500), the computing device 4 will send instructions to the voice identification circuit 23 that it should listen for a word spoken by the user (step 505). The computing device 4 will also give an indication to the user via the display 5 and the speaker 7 that the user is to hold the device in close proximity to his or her lips and say the word that the circuit has been trained for. After the user says the word, the voice identification circuit 23 will send a signal to the computing device 4 that either confirms or denies the correct identity of the user (step 510). If the correct identity is denied, then the computing device 4 will give such an indication to the user via the display 5 and the speaker 7 (step 515), and will then power down (step 516). If the correct identity is confirmed, then the computing device 4 will start an internal count down timer (step 520). If the timer expires before the user starts blowing into the device, then the computing device will indicate an abort situation to the user via the display 5 and the speaker 7, and then power down (step 525). The starting timer value is set short enough as to not allow the user to speak the verifying word and then pass the device to another person for the breath test. As alternate methods, if the correct identity is confirmed, then the computing device 4 will look for: 1) an interruption of the received infrared signal as indicated by the infrared transmitter/receiver circuit 24 before blowing has started; 2) an interruption of the received infrared energy from the passive infrared detector circuit 25 before blowing has started; or 3) the indication of excessive motion as indicated by the motion detector 26 before blowing has started. If there is no abort indication from the appropriate method indicating that the device is being passed to another person, then the breath test will proceed as described above (step 530).

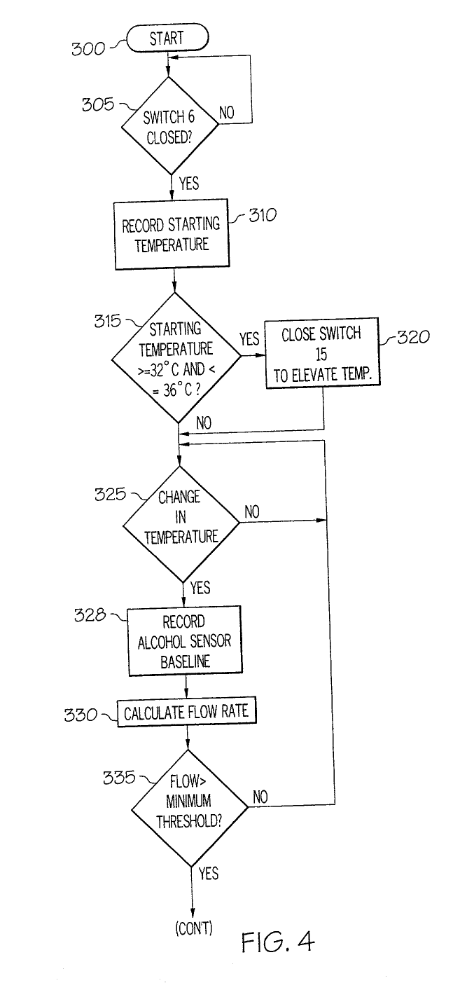

In yet another embodiment of the present invention, the personal breath tester is to be used as an interlock device for the consumer market. FIG. 3 depicts a master transmitter device 60 utilized in the present embodiment which overrides the ignition interlock system 65. It consists of a computing device 27 connected to a wireless transmitter 28 and also to switch 29 and switch 35. When the user presses on the switch 29, the computing device 28 sends a bypass code to the transmitter 28. The ignition interlock system 65 of FIG. 2, which is mounted in the vehicle, receives the bypass code by way of the wireless receiver 19. When the computing device 20 detects the bypass code, it turns on relay 21 to enable the ignition and to allow starting of the vehicle. The bypass code also puts the computing device 20 into a state wherein it will recognize the activation of any number of switches 30 attached to the computing device. The switches 30 represent programming options, such as whether or not a breath test will be required of the user while the vehicle is running. In this manner, the supervisor can program various options into the interlock that the normal user cannot access. In one embodiment of the present invention, the consumer interlock may record a violation, meaning that a breath test was not taken and passed when requested either before starting the vehicle or after the vehicle was running. If this occurs,-the violation will be recorded. Pressing switch 35 on the master transmitter will reset the violation.

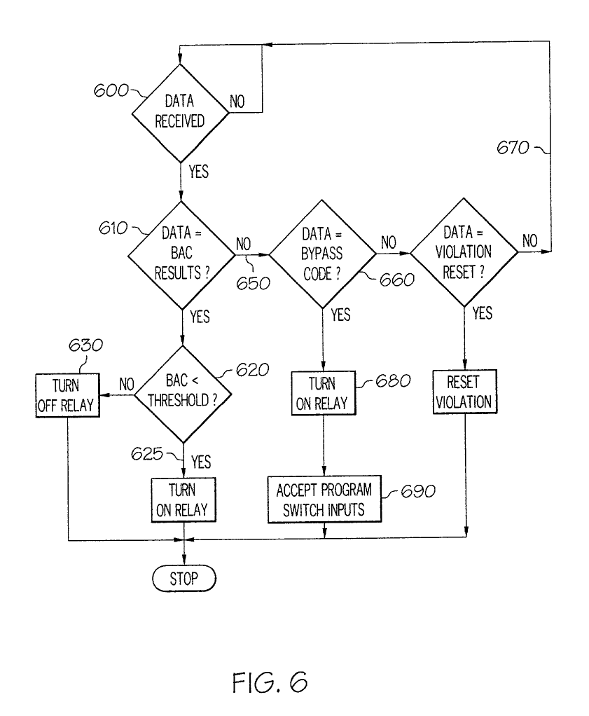

An exemplary method of programming the consumer ignition interlock system 65 is depicted in FIG. 6. The computing device 20 continuously monitors the wireless receiver 19 to determine if any data has been received (step 600). If data is received, the computing device 20 determines whether the data is blood alcohol content results (step 610). If the data is blood alcohol content results, the computing device 20 determines whether the results exceed the threshold (step 620). If the results are less than the threshold, the relay 21 is engaged allowing the vehicle to be started (step 625). If the results exceed the threshold, the relay remains “off” preventing the vehicle from being started (step 630).

If the data received does not contain blood alcohol content results (step 650), the computing device 20 determines whether the data contains a bypass code (step 660). If the data does not contain a bypass code, the computing device 20 clears the data and returns to continuously monitoring the wireless receiver 19 (step 670).

If the data received does contain a bypass code, the relay 21 is engaged (step 680). In a further embodiment of the present invention, the switch 30 of the ignition interlock system 65 can be utilized to reconfigure the program options of the ignition interlock system 65 (step 690).

One skilled in the art will appreciate the various components of the personal breath tester may be obtained from a multitude of sources known to those skilled in the art. For example, ethanol fuel cell sensors may be obtained from Guth Laboratories of Harrisburg, Pa. and from Draeger Safety of Houston, Tex. Typical microprocessors that may be utilized in the present invention may be obtained from Texas Instruments of Dallas, Tex. and NEC of Santa Clara, Calif. Temperature sensors utilized in the present invention may be obtained from NIC of Melville, N.Y. and Murata of Smyrna, Ga. Typical wireless transmitters/receivers which may be utilized in the present invention may be obtained from Atmel of Heilbronn, Germany and RF Microdevices of Greensboro, N.C. Voice identification circuitry may be obtained from Sensory Circuits of Santa Clara, Calif.

The foregoing description of the exemplary embodiments has been presented for purposes of illustration and description. It is not intended to be exhaustive nor to limit the inventor to the precise form disclosed. Obvious modifications or variations are possible in light of the above teachings. The embodiments were chosen and described in order to best illustrate the principles of the invention and its practical application to thereby enable one of ordinary skill in the art to best utilize the invention in various embodiments and with various modifications as are suited to the particular use contemplated. It is intended that the scope of the invention be defined by the claims appended hereto.

We claim:

1. An apparatus for detecting gaseous component levels in breath, comprising:
a) a breath channel;
b) an electrochemical sensor in fluid communication with the breath channel;
c) a processor in electrical communication with the electrochemical sensor; and
d) computer readable storage medium in electrical communication with the processor, wherein the computer readable storage medium contains executable instructions for the processor;
wherein the apparatus is configured to calculate approximate
gaseous components levels in a breath without utilizing a sampling pump.

2. The apparatus of claim 1, further comprising:
a temperature sensor in fluid communication with the breath channel.

3. The apparatus of claim 1, wherein the electrochemical sensor is an ethanol sensor and the gaseous component is alcohol.

4. A breath detection apparatus for detecting gaseous component levels in breath, comprising:
a gas sensor;
a processor in electrical communication with the gas sensor; and
a computer readable storage medium in electrical communication with the processor, wherein the computer readable storage medium contains executable instructions for the processor; and
a wireless transmitter;
wherein the wireless transmitter transmits a signal to an external receiver.

5. The breath detection apparatus of claim 4, further comprising:
a temperature sensor in electrical communication with the processor.

6. The breath detection apparatus of claim 4, wherein the gas sensor comprises an electrochemical fuel cell.

7. The breath detection apparatus of claim 4, wherein the gas sensor comprises an ethanol sensor.

8. An apparatus for detecting gaseous component levels in breath, comprising:
a) a breath channel;
b) a gas sensor in fluid communication with the breath channel;
c) a processor in electrical communication with the gas sensor;
d) computer readable storage medium in electrical communication with the processor, wherein the computer readable storage medium contains executable instructions for the processor; and
e) a temperature sensor in fluid communication with the breath channel;
wherein the temperature sensor is utilized to determine breath flow rate.

9. A method for detecting gaseous component levels in breath comprising the steps of:
providing the apparatus of claim 8;
obtaining an initial signal from the temperature sensor;
monitoring the temperature sensor for a temperature change;
calculating airflow rate utilizing the temperature sensor signal; and
calculating gaseous component levels in breath utilizing airflow rate.

10. The method of claim 9, further comprising the steps of:
determining whether the initial signal is within predefined limits;
if initial temperature is not within predefined limits, heating temperature sensor to elevate output signal from the temperature signal to an acceptable level.

11. The method of claim 9, further comprising the steps of determining whether the gaseous component level exceeds a predefined threshold and transmitting a signal to an interlock device.

12. A method for detecting gaseous component levels in breath comprising the steps of:
providing the apparatus of claim 4;
calculating the gaseous component levels in breath; and
transmitting a signal to an external receiver.

13. A method for detecting gaseous component levels in breath comprising the steps of:
providing the apparatus of claim 1;
receiving a breath stream in the breath channel;
obtaining a signal from the electrochemical sensor
calculating a gaseous component level in breath utilizing the electrochemical sensor.

14. An apparatus for detecting gaseous component levels in breath, comprising:
a breath passage having a flowpath, a proximal end and a distal end, wherein the proximal end comprises an inlet for accepting a person's breath and the distal end comprises an outlet for venting the breath;
a temperature sensor in fluid communication with the flowpath;
an electrochemical sensor in fluid communication with the flowpath;
a processor in electrical communication with the temperature sensor and the electrochemical sensor; and
a computer readable storage medium in electrical communication with the processor, wherein the computer readable medium contains executable instructions for the processor;
wherein the apparatus is configured to approximate gaseous component level in the breath without utilizing a sampling pump.

15. The apparatus of claim 14, further comprising:
a start switch in electrical communication with the processor.

16. The apparatus of claim 14, further comprising a wireless transmitter.

17. An apparatus for detecting gaseous component level in breath, comprising:
a) a breath channel;
b) a gas sensor in fluid communication with the breath channel;
c) a processor in electrical communication with the gas sensor;
d) computer readable storage medium in electrical communication with the processor, wherein the computer readable storage medium contains executable instructions for the processor; and
wherein the apparatus is configured to approximate gaseous component level in a breath without utilizing a sampling pump; and further wherein the apparatus is configured to cease functioning at a pre-determined time.

18. An apparatus for detecting gaseous component level in breath, comprising:
a) a breath channel;
b) a gas sensor in fluid communication with the breath channel;
c) a processor in electrical communication with the gas sensor;
d) computer readable storage medium in electrical communication with the processor, wherein the computer readable storage medium contains executable instructions for the processor; and
wherein the apparatus is configured to approximate gaseous component level in a breath without utilizing a sampling pump; and further wherein the apparatus is configured to cease functioning outside a pre-determined temperature range.

19. An ignition interlock system, comprising:
the breath detection apparatus of claim 4 and
a wireless receiver;
a computing device in electrical communication with the wireless receiver;
a computer readable storage medium in electrical communication with the computing device, wherein the computer readable medium contains executable instructions for the computing device;
a switch in electrical communication with the computing device and ignition control line of a vehicle;
wherein the wireless receiver is configured to receive signals from the breath detection apparatus.

20. The ignition interlock system of claim 19, further comprising:
one or more switches, wherein the switches are configured to allow additional functionality to the ignition interlock system.

21. The ignition interlock system of claim 13, further comprising:
a master override device, wherein the master override device is configured to allow a user to enable starting of the vehicle without taking a breath test or to select one or more override options.

22. The ignition interlock system of claim 13, further comprising:
a voice identification component in electrical communication with the computing device, wherein the voice identification component is configured to verify the user of the device.

23. An identification system for a breath detection interlock system; comprising:
a wireless transmitter and receiver;
a processor in electrical communication with the transmitter and receiver;
a computer readable medium in electrical communication with the processor, wherein the computer readable storage medium contains executable instructions for the processor; and
wherein the executable instruction comprise instructions to maintain a continuous signal between the transmitter and receiver.

24. An identification method for a breath detection system, comprising the steps of:
providing the system of claim 23;
confirm the user's identity;
maintain a continuous signal between the transmitter and receiver after the identity has been confirmed; and
if the signal between the transmitter and receiver is not continuous, abort the breath detection system and restart the system.

25. An identification system for a breath detection interlock system; comprising:
a passive infrared detector
a processor in electrical communication with the detector;
a computer readable medium in electrical communication with the processor, wherein the computer readable storage medium contains executable instructions for the processor; and
wherein the executable instruction comprise instructions to monitor infrared signals utilizing the passive infrared detector.

26. An identification method for a breath detection system, comprising the steps of:
providing the system of claim 25;
confirm the user's identity;
maintain a continuous signal between the user and the passive infrared detector; and
if the signal received by the passive infrared detector is not continuous, abort the breath detection system and restart the system.

27. An identification system for a breath detection interlock system; comprising:
a motion sensor
a processor in electrical communication with the motion sensor;
a computer readable medium in electrical communication with the processor, wherein the computer readable storage medium contains executable instructions for the processor; and
wherein the executable instruction comprise instructions to monitor the movement of the motion sensor.

28. An identification method for a breath detection system, comprising the steps of:
providing the system of claim 27;
confirm the user's identity;
monitoring the motion sensor output; and
if the signal received by the motion sensor exceeds a pre-determined threshold, abort the breath detection system and restart the system.

29. An identification method for a breath detection system, comprising the steps of:
providing a timer, a processor and a computer readable medium in electrical communication with the processor, wherein the computer readable medium contains executable instructions comprising a countdown timer;
confirm the user's identity; and
initiate the countdown timer executable instructions, wherein if a breath test has not been initiated by the lapse of the count down timer, abort the breath detection system and restart the system.
